(19) United States
(12) Patent Application Publication
(10) Pub. No.: US 2012/0183674 A1
(43) Pub. Date: Jul. 19, 2012

(54) Method of Selectively Applying an Antimicrobial Coating to a Medical Device or Device Material

(76) Inventors: Nathan G. Bonn-Savage, Portland, OR (US); Jon N. Neese, Oregon City, OR (US)

(21) Appl. No.: 13/336,193

(22) Filed: Dec. 23, 2011

Related U.S. Application Data
(60) Provisional application No. 61/433,647, filed on Jan. 18, 2011.

(51) Int. Cl. B05D 5/00; B05D 1/02; B05B 15/00; B05D 3/00; B05B 1/00; B82Y 5/00; B82Y 30/00

(52) U.S. Cl. 427/2.1; 427/372.2; 427/256; 118/300; 118/696; 977/773; 977/931

(57) ABSTRACT

A process for depositing nanoparticles on a surface. The process includes the steps of: providing a sol including a volatile non-aqueous liquid and nanoparticles suspended in the non-aqueous liquid; processing the sol to form a plurality of droplets; depositing the plurality of droplets on a surface; and evaporating the non-aqueous liquid from the surface leaving a residue of nanoparticles. The liquid can be selected from heptane, chloroform toluene, and hexane and mixtures thereof and the nanoparticles are desirably silver nanoparticles. The plurality of droplets may be formed by a spray process. The surface may be selected from a particular area, region, portion, or dimension of a medical device, device material, packaging material or combinations thereof. The residue of nanoparticles desirably provides antimicrobial properties.

This application claims the benefit of priority from U.S. Provisional Application No. 61/433,647 filed on Jan. 18, 2011.

### FIELD OF THE INVENTION

The invention relates to a method for preparing liquid mixtures that contains silver nanoparticles. More particularly, the invention relates to silver nanoparticle mixtures for coating purposes and methods for applying mixtures to yield a coating onto portions or the entirety of a medical device, device surface, or material surface.

### BACKGROUND OF THE INVENTION

Application of antimicrobial agents such as metal nanoparticles or antibiotic coatings to surfaces such as, for example, surfaces of medical devices or other material surfaces are typically conducted in a batch style process due to difficulty in maintaining reagent stability and coating uniformity in continuous processes. Exemplary batch style processes may include vapor deposition, direct incorporation of the antimicrobial agent in a material forming the surface, dipping of the device into a bath containing the active agent and a binder material, or a combination of the above processes. Existing methods typically cannot be adapted to continuous or in-line processes and can include the incorporation of expensive equipment, operator skill, and labor intensive steps, Also certain substrates provide a particular challenge in that they require selective application on detailed geometries or are porous and have a requirement that the application be limited as to the depth of impregnation. Currently available dipping processes for the application of coating agents are difficult to implement and generally provide coatings of insufficient concentration tolerances for the desired application herein.

A typical dip type coating can apply silver, Ag, to the surface of a material, but the process is relatively uncontrolled and variable. An example illustrating the variability of results from a dip coating process is shown in FIG. 1 which is a graph of silver deposition expressed in units of micrograms per square centimeter on the y-axis and the number of dips on the x-axis. More particularly, the item dipped was an expanded polytetrafluoroethylene (ePTFE) vascular graft. The graft was deposited in a liquid bath containing a silver nanoparticle and heptane mixture. Each dip or immersion of the article was timed to last for 30 seconds. The sample was air-dried for 5 minutes between dips. The silver deposition was measured utilizing flame atomic absorption spectrophotometry (FAAS).

As is evident from FIG. 1, the number of dips did not correlate well with a predictable or generally uniform increase in the density of silver on the surface.

Accordingly, there is a need for a coating process that can be tightly controlled to provide a relatively predictable and uniform deposition of a metal nanoparticle such as silver nanoparticle. There is also a need for a process that allows selective application of an antimicrobial nanoparticle, flexibility of delivery vehicle (meaning a variety of organic solvents can be employed depending on substrate material), and coating concentration. Moreover, there is a need for silver-containing, non-aqueous formulations that can be the basis of a coating process that is flexible and provides a controllable and relatively predictable and uniform deposition of silver nanoparticles.

### SUMMARY OF THE INVENTION

The present invention addresses the problems described above by providing a method of depositing silver nanoparticles on surfaces. For example, the present invention relates to methods, processes and liquid formulations for depositing silver nanoparticles on surfaces such as, for example, surfaces of medically relevant materials or articles to render them antimicrobial.

According to an aspect of the invention, the process involves providing a sol composed of a volatile non-aqueous liquid and nanoparticles suspended in the non-aqueous liquid. The sol may be provided by preparing an aqueous suspension of nanoparticles and extracting the nanoparticles into a non-aqueous liquid to form a sol. For example, the sol may be prepared by forming an aqueous suspension of silver nanoparticles and extracting the silver nanoparticles into a non-aqueous liquid. Any water immiscible organic solvent may be used in the extraction process.

The sol desirably has low viscosity and is adapted to forming droplets utilizing conventional droplet forming techniques. The sol is then processed to form a plurality of droplets. These droplets are deposited on a surface. Finally, the non-aqueous liquid is evaporated from the surface to leave a residue of nanoparticles. Alternatively and/or additionally to forming droplets, it is contemplated that the process may deposit the sol on a surface by techniques selected from printing, dipping, brushing or combinations thereof.

Generally speaking, the volatile non-aqueous liquid component of the sol may be any water immiscible organic solvent that has a sufficiently low viscosity for an application process such as spraying has a high volatility to be quickly evaporated, is compatible with the nanoparticles, and can be readily handled in an application process. For example, the liquid may be selected from benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform, toluene, and hexane and mixtures thereof. Desirably, the nanoparticle component of the sol is silver nanoparticles. The silver nanoparticles may have an effective diameter of less than 20 nanometers (nm). Even more desirably, the residue of nanoparticles (i.e., the nanoparticles deposited on the surface) provides antimicrobial properties. It is contemplated that the sol may further include other materials having antimicrobial properties including, but not limited to, copper nanoparticles, chlorohexidine, iodine, antibiotics and combinations thereof.

The plurality of droplets may be formed by a spray process. For example, the spray process may utilize a centrifugal pressure nozzle, a solid cone nozzle, a fan spray nozzle, a sonic atomizer, a rotary atomizer, a flashing liquid jet, ultrasonic nozzles or combinations thereof. The spray process may utilize electrostatic charge. The surface to be treated may be a particular area, region, portion, or dimension of a medical device, device material, packaging material or combinations thereof.

In an aspect of the invention, the steps of depositing the plurality of droplets on a surface and evaporating the non-aqueous liquid from the surface leaving a residue of nanoparticles may be conducted a plurality of times. According to the invention, the process may deposit nanoparticles on a porous surface such that the nanoparticles penetrate the porous surface. More particularly, the process may deposit nanoparticles on a porous surface in such manner that the penetration of nanoparticles into the porous surface is controlled.

The present invention encompasses a system for depositing nanoparticles on a surface. The system includes: (i) a spray coating device including a spray head for spraying a metal nanoparticle sol; and (i) a nanoparticle sol including 25 to 5000 parts per million of metal nanoparticles; and 995000 to 999975 parts per million of a non-aqueous liquid, wherein the metal nanoparticle sol has a viscosity of about 1 Centipoise (cP) or less at 25° C.

The system may include a booth including an exhaust system to remove volatile organic vapors. The system may also include an automated programmable coating counter to control a number of spray coats and a point of shut-off for the spray head. According to the system, the non-aqueous liquid may be benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform toluene, and hexane and mixtures thereof. The nanoparticles desirably have an effective diameter of less than 20 nm and, more desirably, are silver nanoparticles.

The present invention also encompasses an article including a surface containing nanoparticles deposited according to any of the above-described processes or system. Desirably, the nanoparticles are present at only the article surface. Even more desirably, the nanoparticles are silver nanoparticles.

Other objects, advantages and applications of the present disclosure will be made clear by the following detailed description.

### DESCRIPTION OF THE DRAWINGS

FIG. 1 is an illustration of a graph of silver deposition provided by a conventional dip process. The silver deposition is expressed in units of micrograms per square centimeter on the y-axis and the number of dips on the x-axis.

FIG. 2 is a schematic view illustration showing an exemplary apparatus used in a process for deposition of nanoparticles.

FIG. 3A is a left side view illustration showing an exemplary spray head of an exemplary apparatus shown in FIG. 2 used in a process for deposition of nanoparticles.

FIG. 3B is a front view illustration showing an exemplary spray head of an exemplary apparatus shown in FIG. 2 used in a process for deposition of nanoparticles.

FIG. 3C is a top view illustration showing an exemplary spray head of an exemplary apparatus shown in FIG. 2 used in a process for deposition of nanoparticles.

FIG. 4 is an illustration of a graph of silver deposition provided by an exemplary process for deposition of nanoparticles as illustrated in FIGS. 2 and 3. The silver deposition is expressed in units of micrograms per square centimeter on the y-axis and the number of spray passes on the x-axis.

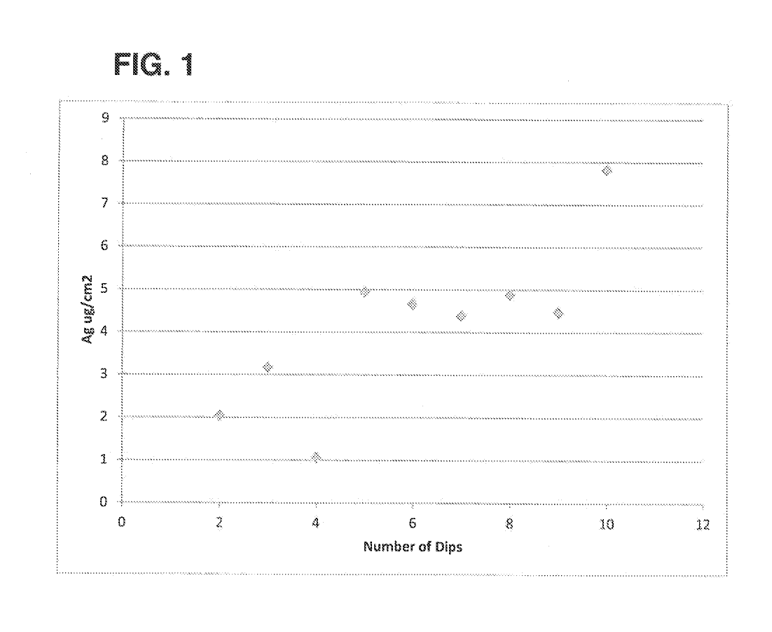

### DETAILED DESCRIPTION

To illustrate the invention and demonstrate its operation, various articles were prepared by applying silver nanoparticles (occasionally referred to herein as “nanosilver”) onto selective surfaces of various materials. However, it is contemplated that the metal nanoparticle may be gold, platinum, indium, rhodium, palladium, copper or zinc. The nanoparticles may be in the size range of 0.1 to 100 nm. These nanoparticles may have a standard normal size distribution; however, nanoparticles less than about 20 nm have been found to work well.

The silver nanoparticles were applied or deposited onto surfaces from a sol composed of a volatile non-aqueous liquid and nanoparticles suspended in the non-aqueous liquid. The sol may be readily provided by preparing an aqueous suspension of nanoparticles and extracting the nanoparticles into a non-aqueous liquid to form a sol. Suitable techniques may be found at, for example, U.S. Patent Application Publication No. 2007/0003603 for “Antimicrobial Silver Composition” published Jan. 4, 2007, the contents of which are incorporated herein by reference.

Generally speaking, the liquid component of the sol is any volatile water immiscible organic solvent that has a sufficiently low viscosity for the application process (e.g., spraying), has a relatively high volatility to be quickly evaporated, is compatible with the nanoparticles, and can be readily handled in an application process. For example, the liquid may be selected from benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform, toluene, and hexane and mixtures thereof. Silver nanoparticles having an effective diameter of less than 20 nm have been found to work well. A silver nanoparticle sol having a viscosity of about 1 cP or less at 25° C. has been found to work well. The viscosity of the nanoparticle sol at the typical concentrations of nanoparticles (e.g., 25 to 5000 parts per million) will have a viscosity of the volatile water immiscible organic solvent. Of course, the viscosity may be determined utilizing viscometers such as a Brookfield RV DV-E Viscometer with Helipath Spindle Set (T-bar spindles). However, the viscosity may be so low that it may be only possible to determine that the viscosity is less than 1 cP with conventional viscometers.

The surface to be treated may be a particular area, region, portion, or dimension of a medical device, device material, packaging material or combinations thereof. The surface may be non-porous or porous. Desirably, the surface may be porous or have a surface texture or topography.

In an aspect of the invention, the steps of depositing the plurality of droplets on a surface and evaporating the non-aqueous liquid from the surface leaving a residue of nanoparticles may be conducted a plurality of times. According to an aspect of the invention, the process may deposit nanoparticles on a porous surface (e.g., an expanded material such as expanded polytetrafluoroethylene) such that the nanoparticles penetrate into the porous surface. More particularly, the process may deposit nanoparticles on a porous surface in such manner that the penetration of nanoparticles into the porous surface is controlled. This can be important in a variety of applications where nanoparticles are desired to be present at or near a surface (e.g., beneath a surface) but not penetrated entirely through or throughout a material.

The present invention encompasses a silver nanoparticle sol composed of 25 to 5000 parts per million of silver nanoparticles; and 995000 to 999975 parts per million of a non-aqueous liquid. For purposes of the present invention, a concentration of nanoparticles in non-aqueous characterized as 1,000 parts per million (i.e., 1,000 parts nanoparticles to 1,000,000 parts non-aqueous liquid) generally correspond to 1,000 micrograms (μg) of nanoparticles per 1,000,000 grams (g) of liquid which may be expressed as (μg/g). In other words, a nanoparticle concentration of 1 part per million (i.e., 1 ppm) generally corresponds to a concentration of 1 μg/g for the types of nanoparticles and non-aqueous liquids employed in the present invention. Desirably, the silver nanoparticles have an effective diameter of less than 20 nm. The silver nanoparticle sol also has a viscosity of about 1 cP or less at 25° C. The non-aqueous liquid may be benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform, toluene, and hexane and mixtures thereof.

The sol desirably has low viscosity and is adapted to forming droplets utilizing conventional droplet forming techniques. The sol is then processed to form a plurality of droplets utilizing conventional spray processes or techniques. For example, a spray process may utilize a centrifugal pressure nozzle, a solid cone nozzle, a fan spray nozzle, a sonic atomizer, a rotary atomizer, a flashing liquid jet, ultrasonic nozzles or combinations thereof. The spray process may utilize electrostatic charge.

These droplets are deposited on a surface. Alternatively and/or additionally to forming droplets, it is contemplated that the process may deposit the sol on a surface by techniques selected from printing, dipping, brushing or combinations thereof. The surface to be treated may be a particular area, region, portion, or dimension of a medical device, device material, packaging material or combinations thereof. The surface may be hydrophobic or hydrophilic. The surface (or portions of the surface) may be pretreated to modify the surface energy to enhance the application of the sol or to help repel the sol. Non-polar non-aqueous liquids such as, for example, heptanes have been found to work particularly well on hydrophobic surfaces such as, for example, polytetrafluoroethylene.

After the sol is deposited on the surface, the non-aqueous liquid is evaporated from the surface to leave a residue of nanoparticles. A spray booth or similar structure with an exhaust system is useful to provide a flow of air to help evaporate the non-aqueous liquid and to properly handle the vapor. The residue of nanoparticles adheres to the surface of the article. The steps of depositing the sol (e.g., as a plurality of droplets or by other techniques) on a surface and evaporating the non-aqueous liquid from the surface leaving a residue of nanoparticles may be conducted a plurality of times.

The residue of nanoparticles may be designed to provide antimicrobial properties. Desirably, the nanoparticles are present at only the article surface. It is contemplated that the sol may further include other antimicrobial constituents including, but not limited to, copper nanoparticles, chlorohexidine, iodine, antibiotics and combinations thereof to enhance the antimicrobial properties of the residue.

In one example, polytetrafluoroethylene material was treated selectively on the outer dimension of a tubular structure with nanoparticles of antimicrobial silver suspended in heptane, chloroform, and toluene, or mixtures thereof, by a spray technique utilizing a spray apparatus. In other examples, the nanoparticles have been applied to the surface of polytetrafluoroethylene material by dipping, brushing, or dripping the solvent/nanosilver mixture onto the surface of the material. Other examples represent additional materials that have been imparted with nanosilver in this fashion including silicone, paper, polyethylene, polystyrene, Styrofoam, polypropylene, wood, cotton, and polycarbonate. The nanosilver used in these examples is initially generated as an aqueous suspension according to commonly assigned U.S. Patent Application Publication No. 2007/0003603 for “Antimicrobial Silver Composition” published Jan. 4, 2007, the contents of which are incorporated herein by reference. U.S. Patent Application Publication No. 2007/0003603 corresponds to PCT/US2005/027261 and PCT International Application Publication WO2006026026A2). The silver nanoparticles generated in the aqueous suspension are then subjected to an extraction step that includes the total transfer of nanosilver from the aqueous phase into the organic phase of choice (e.g., heptane, chloroform and/or toluene).

### EXAMPLES

### Example 1

### Selective Spray Deposition on Polytetrafluoroethylene (PTFE)

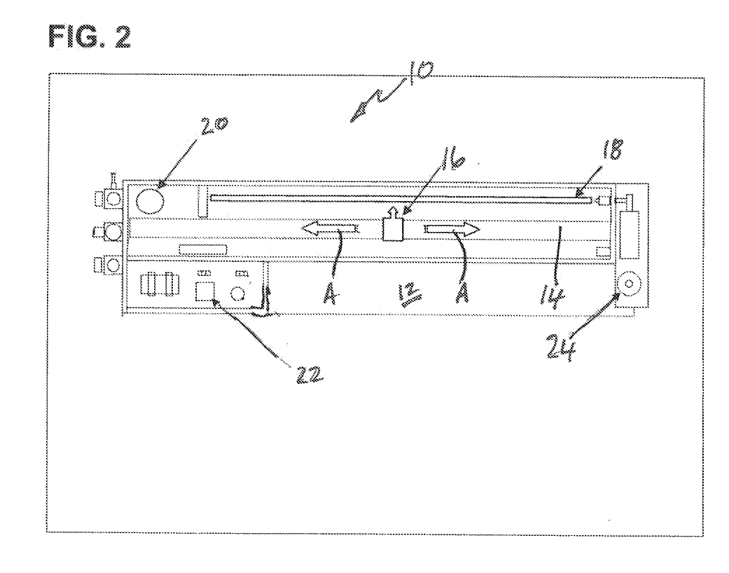

It was desired to deposit nanosilver selectively to the outside diameter of a tubular structure. A spray deposition technique was developed to deposit silver in such a manner as to uniformly apply a coating on the outside of the tubular expanded PTFE or ePTFE (expanded polytetrafluoroethylene is available from W.L. Gore & Associates) material while leaving the inside diameter completely free of silver. The ePTFE graft material treated in this example was a hollow tube with an internal diameter of 6 mm and a length of up to 44 inches. The uniform application of the nanosilver was accomplished by rotating the tubular material on a mandrel that spans the length of the tubular structure. Referring to FIG. 2 of the drawings, there is shown a schematic drawing of an automated apparatus 10 for spraying the length of a tubular structure uniformly. The apparatus includes a base 12, a track 14 for a spray head 16 that can move along the track in the directions of the arrow “A” associated therewith. Parallel to the track 14 and in range of the spray head 16 is a mandrel 18 that is adapted to hold a tube or similar article. The mandrel 18 is configured to rotate. Rotation of speeds of between 500 and 4000 revolutions per minute (RPM) have been found to provide satisfactory results. The examples were produced at rotation speeds of about 3000 RPM.

This equipment could also utilize multi-axis motion control to precisely control the application of nanoparticles to complex substrate geometries. The nanoparticle sol may be contained in a reservoir 20. It is contemplated that the nanoparticle sol may be fed from an external reservoir. Features including a spray pass counter 22, motor controls 24, regulators for spray control, spray head position, and the like may be included.

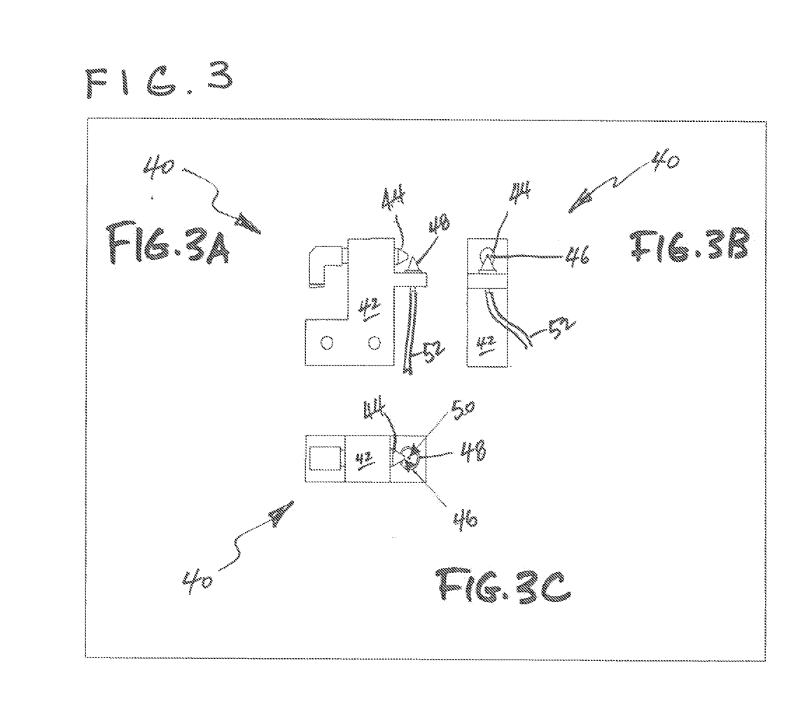

Referring to FIGS. 3A-C, there is shown an exemplary spray head utilized in the spray apparatus illustrated in FIG. 2. FIG. 3A is a side view of a modified Venturi spray head 40. More particularly, FIG. 3A is a view of the side of the spray head located on the left side when the spray head is viewed from the front. FIG. 3B is a front view of the modified Venturi spray head 40. More particularly, FIG. 3B is a view of the front face or front side of the spray head. FIG. 3C is a top view of the modified Venturi spray head 40. The spray head 40 includes mount 42 that supports a first housing 44 defining a first orifice 46 (referred to as an air or gas orifice 46—although gases such as, for example, nitrogen, carbon dioxide, argon or the like may be used instead of or in combination with air) for the supply of pressurized gas. The mount 42 of the spray head 40 also supports a second housing 48 defining a second orifice 50 (referred to as a Venturi orifice 50). A small diameter tube 52 is submerged into nanoparticle sol (not shown) in order to transfer the nanoparticle sol to the spray head 40 that sprays the mixture onto the intended substrate—which is desirably mounted on the mandrel 18. The Venturi orifice 50 is located in the path of the stream of gas exiting the gas orifice 46. Due to the pressure difference, the nanoparticle sol is drawn through the Venturi orifice 50 and into the moving gas flow exiting the gas orifice 46. The nanoparticle sol is projected as a fine spray of droplets onto the article mounted on the mandrel 18.

The spray coating was conducted in a specially designed and fabricated spray booth that included multi-axis spraying capabilities, specialized exhaust features to remove volatile organic vapors, and an automated programmable coating counter to control the number of spray coats and the point of shut-off for the spray head.

### Process:

This treatment process includes the following steps:
1. Formation of aqueous Ag nanoparticles (AgNP) mixture. This step involves the typical batching of a silver nanoparticle recipe (See U.S. Patent Application Publication No. 2007/0003603 for “Antimicrobial Silver Composition”). The preparation is summarized below:
   1 part by volume of ‘1×’ (16.67 g/L) Tween 20 surfactant (=Polysorbate 20 or polyoxyethylene (20) sorbitan monolaurate)
   1 part by volume 0.05M Sodium Acetate
   1 part by volume 0.15M Silver Nitrate
   Mixture is heated to ˜55 C
   1/10 part by volume of N, N, N′, N′ tetramethylethylenediamine (TEMED).
   Mixture is maintained at ˜55 C for 16+ hours.
2. Extraction of AqNP into Heptane to form AgNP:Heptane mixture. This step involves the destabilization of AgNP and re-dispersion into heptane.
   AgNP mixture is maintained at 55 C.
   Na Citrate is added to make the solution 2M (516 g/L). (A 7:3 volume ratio of AgNP:99% Isopropyl Alcohol (IPA) can also be used).
   Mixture is allowed to cool to room temperature under stirring. A brown to black oily precipitate will form.
   The aqueous layer is decanted, leaving behind the oily precipitate containing AgNP.
   An equal volume of heptane, chloroform, toluene, or mixtures thereof is added and stirred for up to 16 hours. The AgNP will re-disperse in this liquid, making it amber to brown in appearance.
   The organic layer is then decanted and filtered, leaving behind the oily precipitate.
   The concentration of this suspension can be monitored using UV/vis spectrophotometry at the 420 nm wavelength. A typical mixture will be diluted 1:3 with heptane and the absorbance at 420 nm recorded. The desired absorbance of this diluted mixture will be 1.5AU. The Ag nanoparticles are thus suspended in heptane.
3. Treatment of ePTFE Material. This step involves the actual coating of the ePTFE material in the AgNP:Heptane mixture.
   The tubular ePTFE material is placed on provided stainless steel mandrels and stretched as completely as possible (i.e., without causing permanent deformation of or damage to the material). Stretching allows for a uniform coating of the ePTFE which is a very pliable and soft substrate. Without stretching the resulting coating is visually non-uniform. The mandrels must be dry and at no time are the mandrels or grafts to be handled with ungloved hands. The mandrels also prevent inadvertent spray treatment of the lumen of the tubular material with nanoparticles.
   The appropriate amount of AgNP:Heptane mixture is poured into a reservoir to supply the spray apparatus.
   The desired number of spray coatings is selected and the coating is performed.

After the ePTFE material was coated with silver, it was tested for antimicrobial efficacy utilizing a conventional 24 hour bacterial challenge assay. In such a test, the substrates are challenged with known bacterial count while immersed in medium for 24 hours. The medium was then appropriately diluted and plated on MHA (Mueller-Hinton Agar) plates to estimate the surviving bacterial count. A log reduction of bacteria exposed to the treated substrate over a 24-hour period is a typical test to measure antimicrobial activity. A reduction of 3-logs (99.9%) of bacteria is widely considered to indicate a coating or treatment that is highly effective as an antibacterial agent. Table A demonstrates the antimicrobial nature of the deposited nanosilver against Methicillin Resistant Staphylococcus Aureus (MRSA). In Table 1, T0 is the zero time inoculum and T1 is 24 hour time survivor count. The log T0 data is included to confirm that nothing was abnormally affecting bacterial growth on the untreated plates. The data in Table A below indicate a log reduction in excess of the 3-log threshold.

TABLE A
Demonstration of Antimicrobial Nanosilver Coating on PTFE against
MRSA

| | | | | Untreated Control | | |
|---|---|---|---|---|---|---|
| | | | | Substrate | | |
| | Silver Treated Substrate | | | (ID: AI 29507) | | |
| | (n = 3) | | | (n = 2) | | |
| 24-Hour | | | Log₁₀ | Log₁₀ | Log₁₀ | Log₁₀ |
| Samples | Log₁₀ T₀ | Log₁₀T1 | Reduction | T₀ | T1 | Reduction |
| 0111-21A | 4.93 | 1.00 | 6.22 | 4.93 | 7.22 | — |
| 0111-21B | 4.84 | 2.48 | 4.78 | 4.84 | 7.26 | — |
| 0111-21C | 4.84 | 2.51 | 4.75 | 4.84 | 7.26 | — |

T₀: Zero time inoculum,
T1: 24-hour time survivor count
*Log reduction = Log₁₀(Untreated Control Substrate at T1) − Log₁₀ (Treated Substrate at T1)

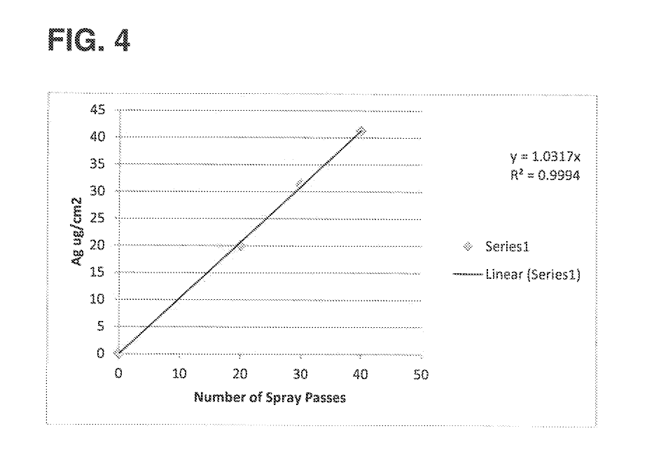

FIG. 4 illustrates the relative uniformity and predictability of results from the spray coating process described above in this Example 1. FIG. 4 is a graph of silver deposition expressed in units of micrograms per square centimeter on the y-axis and the number of spray passes on the x-axis. More particularly, the ePTFE tube was sprayed for approximately 20 seconds and was allowed to air dry for 30 seconds between each spray. The silver deposition was measured utilizing flame atomic absorption spectrophotometry (FAAS).

### Example 2

### Selective Nanosilver Deposition onto Paper and Other Materials by Brushing or Dripping

Paper of various constructions, including notebook paper, cardboard, particulates, was treated with nanosilver by dripping a mixture of an organic solvent and suspended nanoparticles onto a selected surface of material. This was conducted using chloroform, toluene, and heptane as the solvent or combinations thereof and nanosilver as the nanoparticles. The volatile nature of these solvents allows the solvent to evaporate before the untreated side of the substrate is saturated and therefore allows silver to be deposited only on one side of the paper. This method was also performed on materials made with polyethylene, polystyrene, Styrofoam (using only heptanes), polypropylene, wood, cotton (such as a gauze material), and polycarbonate. The advantage of solvent based nanosilver deposition is the rapid nature of the deposition time and the selectivity of the treatment method to render materials antimicrobial.

It will be recognized that the above methods and examples can be modified as appropriate without departing from the scope of the invention. The silver deposition step may be carried out at room temperature or optionally below or above room temperature. The substrate to be coated with nanosilver can undergo identical spray, dip, or brushing steps to increase the surface concentration of nanosilver as desired. Additionally, it has been verified that the AgNP:Organic mixture can be stored in excess of 6 months, the nanosilver particles remain uniformly suspended in the mixture, and the mixture remains viable for the coating process.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

We claim:

1. A process for depositing nanoparticles on a surface, the process comprising:
providing a sol comprising a volatile non-aqueous liquid and nanoparticles suspended in the non-aqueous liquid;
processing the sol to form a plurality of droplets;
depositing the plurality of droplets on a surface; and
evaporating the non-aqueous liquid from the surface leaving a residue of nanoparticles.

2. The process of claim 1, wherein the liquid is selected from heptane, chloroform toluene, and hexane and mixtures thereof.

3. The process of claim 1, wherein the nanoparticles are silver nanoparticles.

4. The process of claim 1, wherein the plurality of droplets are formed by a spray process.

5. The process of claim 1, wherein the surface is a selected from a particular area, region, portion, or dimension of a medical device, device material, packaging material or combinations thereof.

6. The process of claim 4, wherein the spray process is a spray atomization process.

7. The process of claim 1, wherein the residue of nanoparticles provides antimicrobial properties.

8. The process of claim 1, wherein the sol further includes copper nanoparticles, chlorohexidine, iodine, antibiotics and combinations thereof.

9. The process of claim 1, further comprising the steps of preparing an aqueous suspension of silver nanoparticles and extracting the silver nanoparticles into a non-aqueous liquid to form a sol.

10. The process of claim 1, wherein the steps of depositing the plurality of droplets on a surface and evaporating the non-aqueous liquid from the surface leaving a residue of nanoparticles is conducted a plurality of times.

11. The process of claim 1, wherein the process deposits nanoparticles on a porous surface and the nanoparticles penetrate the porous surface.

12. A process for depositing nanoparticles on a surface, the process comprising:
providing a sol comprising a volatile non-aqueous liquid and nanoparticles suspended in the non-aqueous liquid;
depositing the sol on a surface; and
evaporating the non-aqueous liquid from the surface leaving a residue of nanoparticles.

13. The process of claim 12, wherein the liquid is selected from heptane, chloroform toluene, and hexane and mixtures thereof.

14. The process of claim 12, wherein the nanoparticles are silver nanoparticles.

15. The process of claim 12, wherein the sol is deposited on a surface by techniques selected from printing, dipping, brushing or combinations thereof.

16. The process of claim 12, wherein the surface is a selected from a particular area, region, portion, or dimension of a medical device, device material, packaging material or combinations thereof.

17. The process of claim 12, wherein the residue of nanoparticles provides antimicrobial properties.

18. The process of claim 12, wherein the sol further includes copper nanoparticles, chlorohexidine, iodine, antibiotics and combinations thereof.

19. The process of claim 12, further comprising the steps of preparing an aqueous suspension of silver nanoparticles and extracting the silver nanoparticles into a non-aqueous liquid to form a sol.

20. The process of claim 12, wherein the process deposits nanoparticles on a porous surface and the nanoparticles penetrate the porous surface.

21. A system for depositing nanoparticles on a surface, the system comprising:
a spray coating device including a spray head for spraying a metal nanoparticle sol; and
a nanoparticle sol comprising:
25 to 5000 parts per million of metal nanoparticles; and
995000 to 999975 parts per million of a non-aqueous liquid,
wherein the metal nanoparticle sol has a viscosity of less than 1 cP at 25° C.

22. The system of claim 21, further comprising a booth including an exhaust system to remove volatile organic vapors.

23. The system of claim 21, further comprising an automated programmable coating counter to control a number of spray coats and a point of shut-off for the spray head.

24. The system of claim 21, wherein the non-aqueous liquid is selected from benzene, butanol, carbon tetrachloride, cyclohexane, 1,2-dichloroethane, dichloromethane, ethyl acetate, ethyl ether, iso-octane, methyl-t-butylether, methyl ethyl ketone, pentane, heptane, chloroform toluene, and hexane and mixtures thereof.

25. The system of claim 21, wherein the nanoparticles are silver nanoparticles.
